(19) United States
(12) Patent Application Publication
(10) Pub. No.: US 2007/0265568 A1
(43) Pub. Date: Nov. 15, 2007

(54) Automatic Injector

(76) Inventors: Izrail Tsals, Newtown, PA (US); Gilad Lavi, Rishon Letzion (IL)

Correspondence Address: Michael W. Ferrell;Ferrells, P.O.Box 312, Clifton, VA, 20124-1706, US

(21) Appl. No.: 11/666,851

(22) PCT Filed: Nov. 4, 2005
(86) PCT No.: PCT/US05/39979

§ 371 Date: May 2, 2007

Related U.S. Application Data
(60) Provisional application No. 60/624,864, filed on Nov. 4, 2004.

(51) Int. Cl. A61M 5/20

(52) U.S. Cl. 604/136; 604/890.1

(57) ABSTRACT

An injector is automatic in that the needle insertion into the injection site (e.g., a patient's skin) is triggered by the user or caregiver, the insertion is automatic, the following delivery is automatically initiated upon needle insertion, and the needle is shielded automatically after the end of delivery. Preferably the user does not see the needle prior to, during, or after injection. The injector includes a proximal and distal housings and a needle shield arranged to slide on the distal housing. The injector has a driver arranged to move the syringe to cause the needle penetration, to inject the drug, and subsequently to release the shield. The syringe needle is shielded and the shield locked automatically after delivery is completed. The patient does not experience any injector force on the tissue during injection. The injector has built in patient safety features.

### FIELD OF THE INVENTION

This invention relates to the preparation and administration of a product into a living organism (e.g. the human body), and more particularly to an apparatus for automatically and safely delivering the product.

### BACKGROUND OF THE INVENTION

Previously, various devices have been developed for the delivery of medications into and through the skin of living organisms. These devices include syringes in which a liquid drug solution is delivered through the skin of a user from a syringe chamber by movement of a syringe plunger to move the drug solution from a syringe chamber through a syringe needle inserted under the skin. The drug solution is generally in liquid form, and can be a mixture of the drug (e.g. powdered, lyophilized, concentrated liquid) and a diluent (e.g. dextrose solution, saline solution, water).

It is well known that many persons are apprehensive of receiving an injection from a needle. This problem is even more significant for those who must administer their own medication. It is known that needle phobia can be minimized by hiding the needle before, during and after delivery. It is therefore preferable that the person who receives the drug should not see the needle, which often triggers the fear of needle insertion.

It is also preferable for the needle to be protected before and after delivery of the drug. While a needle can be protected with a removable cap, it is preferable for the needle to be secured within the delivery device before the needle is inserted through the patient's skin and after the needle is shielded. Preferably the needle is enclosed in the device after use and locked into final position after injection is completed. The needle insertion is automatic after the injector is triggered by the user or caregiver. The drug injection and shielding are automatic, so that the user does not prematurely expose the needle for injection or have to guess when delivery is completed.

It is also preferable for such a device to provide indications for assisting in the correct use of the automatic injector. Indications could be visual, audible or tactile and provided at the start or completion of any stage of system use to assist the user and to increase the user confidence.

A user or patient could be injured if an injection device were activated prematurely. Generally, such a device projects its needle from the end of a barrel and ejects the dose. Such actions can cause injury if the needle pierces another person or is injected into an undesired area of the patient (e.g. an eye). Accordingly, it is advantageous if the needle is in a safe location before and after use to prevent accidental injury or contamination.

It is also desirable to provide a delivery system where the dosage for delivery could be easily viewed by the patient prior and after use. The user's inability to see the dosage form prior to use creates a significant sense of unease in the user in that the user wants to ensure that the proper dosage is in the system and ready for delivery. More importantly, the user's inability to see the dosage form prior to use leaves the user concerned that the dosage may be faulty, have, for example, foreign particles trapped which if present, may result in injury or harm to the user. The user's inability to view the dosage being delivered and the empty syringe at the end of delivery leaves the user with a level of uncertainty as to the amount delivered and the delivery being completed. Thus, it is important to the user's peace of mind to provide an area in which to view the dosage prior to and after delivery. The detailed injection device provides this opportunity. Most drug makers usually instruct the user to check the drug before use, its transparency, its color and its expiration date to make sure the drug is safe and effective for use.

Further, it is desirable to provide a delivery system that is easy to use at a low cost. Moreover, it is desirable to provide a system that is easy to integrate with the drug container providing flexibility in meeting the requirements of different drug containers like pre-filled syringes/cartridges. For example it is important to accommodate standard cartridges and syringes with needle cover including a rigid plastic cap. Moreover, it is desirable to have a system that could accommodate cartridges and syringes filled on standard filling lines. It is desirable to provide a system characterized by a small number of components indicating low product costs.

None of the prior art detailed below discloses or suggests the mechanism of this invention. This invention implements the triggering of the mechanism by pushing on the trigger, automatic insertion of the needle by advancing the syringe, automatic delivery using same driving means, automatic deployment of the needle shield triggered by completion of injection. The device has provision for effectively delivering the full content of the syringe independent of the component tolerances. The simplicity in implementing these functions within the discussed invention is a major difference as compared to other prior art.

U.S. Pat. No. 5,681,291 to Galli discloses a device inserting the needle after the device is triggered and injecting the contents of the syringe. The shielding of the needle is enabled by the insertion of the needle. As a result the shield exerts a shield force on the injection site for the continuation of injection. This force should be low to be tolerated by the patient. The reliability of the shield deployment might be therefore sensitive to shield friction. The shielding occurs when the injector is removed from the site. The design is complex utilizing a number of components.

Gabriel patents (U.S. Pat. No. 5,114,406 and U.S. Pat. No. 6,544,421) disclose a plunger which is telescopically received within a tubular element causing the needle penetration, drug delivery and securing the needle. Being a telescope type mechanism the system is using two springs. The detection of end of delivery is controlled by part dimensions. Similar to the Galli patent the shield exerts a force on the injection site for the continuation of injection. This force should be low to be tolerated by the patient. Shielding occurs when the injector is removed from the site. The design is complex utilizing a high number of components.

A patent to Marshall et al. (U.S. Pat. No. 5,599,309) discloses an injector having a drive member held in a rearward primed position by a detent provided in the body of the device. When the device is applied to a patients skin and a rear end cap is pressed forwardly, the forward ends of ribs wedge tongues inward (or pivot) until they clear the detents formed by the forward ends of the slots. A coil spring shoots a cylinder forward for injection and delivery. This invention is involving the part tolerances in order to detect end of delivery and is using two springs—one to penetrate and deliver and the second to extract the syringe and shield the needle.

Patents to Crossman, et al. (U.S. Pat. No. 6,159,181 and U.S. Patent Application Publication No. 20030093036) are mechanisms to deliver drugs in a parenteral method and to shield the penetrating needle after use. Both mechanisms are using double springs. In 20030093036 Crossman expects the user to manually trigger the needle shielding and to decide when to do that. The evolution between the first and second application is in making the device simpler and more accurate. Nevertheless the basic principles remain the same.

Patents to Kirchhofer (U.S. Pat. No. 6,280,421 and later related patents) describe an injector with a driven member shiftably accommodated by the housing. The driven member forwards the syringe to insert the needle and then injects the syringe contents. The switch from the syringe forwarding to drug injection is based on the syringe position resulting in a complex mechanism. The forwarding of the syringe compresses the shield spring and releases the shield. As a result the shield exerts a force on the injection site for the duration of injection till the auto-injector is removed from the injection site. The shielding occurs during the injector removal. The design is complex utilizing a high number of components.

In the patent application by Lavi et al. published as U.S. Patent Application Publication No. 20030105430 the mechanism of 10 parts is complex. The mechanism performs a combination of slide and rotate move, the end of delivery is detected by the part dimensioning. The design is characterized by high complexity and costs.

The patent application by Amark (U.S. Patent Application Publication No. 20040039336) describes an injector of a complex construction built around a unique syringe. The assembly process is not suited for standard filling lines and common sterilization methods. The first spring when released pushes on the stopper to insert the needle and to deliver the compound. The shield is released by a third activating means. The user sense higher force on the injection site due to the double safety provision in triggering. No consideration is given to minimizing the drug residue in the syringe. Furthermore the device is complex and employs a number of parts.

### SUMMARY OF THE INVENTION

In an exemplary embodiment, an automatic injector for delivering a fluid includes only seven components: a pre-filled syringe, and a proximal housing, a distal housing, a shield, a driver and a driver and shield springs. The proximal housing includes means arranged to retain the driver in storage and to release it during activation of the injector. The drug syringe is positioned within the housing and includes a barrel, a stopper, a rod, a needle extending toward the distal end of the housing and a needle cap. The barrel is arranged to contain a fluid in communication with the needle. The stopper is slidingly located within the barrel for forcing the fluid through the needle upon activation of the injector. The driver is in communication with the proximal housing, the shield, the driver spring and the cartridge.

The auto-injector in this exemplary embodiment requires the user to enable the triggering mechanism by removing the needle cap and bending a tamper evident safety tab. The device is triggered after the injector is applied to the injection site. When disengaged from the proximal housing the driver is arranged to forward the syringe to insert the needle and only then to act on the stopper to the initiate of the injection process and deliver the drug. The syringe advancement, the needle insertion and the injection are driven by the energy of the driver spring.

The injector also includes a mechanism that automatically triggers shielding of the syringe needle close to the end of delivery. The driver releases the spring loaded shield at a point close to the completion of the injection while allowing the shield release account for all potential component dimensional tolerance stack up. The drug remaining in the syringe during the shield release is delivered during the time required to initiate the move of the shield to shield the needle.

As described above the driver releases the shield toward the end of the injection. No force is exerted by the shield on the injection site till the release of the shield. The force of the shield spring applied to the injection site results in the needle extraction and shielding. The shield is automatically moved to a shielded position and is locked. The needle is positioned within safe margin from the end of the shield. An excessive force would be required to overpower the shield retention feature after the shield is placed in the locked, discard position.

Further scope of applicability of the present invention will become apparent in the description given hereafter. However, it should be understood that the detailed description and specific examples, while indicating preferred embodiments of the invention, are given by way of illustration only, since the invention will become apparent to those skilled in the art from this detailed description.

### BRIEF DESCRIPTION OF THE DRAWINGS

The invention will be described in conjunction with the following drawings, in which like-referenced numerals designate like elements, and wherein:

FIG. 1 is an external perspective view showing an injector construed in accordance with an exemplary embodiment of the invention;

FIG. 2 is a longitudinal cross-section view showing an injector constructed in accordance with an exemplary embodiment of the invention in it's storage position illustrating the cartridge axial support by the driver, radial support by the distal housing and trigger and shield locking elements;

FIG. 3 is a view similar to that of FIG. 2, but showing the injector in a state wherein the protective needle cover assembly has been removed, the injector has been applied to an injection site, the device triggered and the driver has forwarded the syringe to insert the needle;

FIG. 4 is a view similar to that of FIG. 2, but showing the injector in a state wherein the injection is almost completed and the driver is releasing the shield;

FIG. 5 is a view similar to that of FIG. 4, but showing the injection completed by the damping element and the shield beginning to move;

FIG. 6 is a view similar to that of FIG. 5, but showing the shield shielding the needle and locked in the discard position;

FIGS. 7A, 7B and 7C are enlarged cross-section views which illustrate the operation of the shield storage and discard latches;

FIG. 8 is an enlarged cross-section view which illustrates the preferred embodiment of the damping spring element;

FIGS. 9A, 9B, 9C are enlarged cross-section views illustrating structure and operation of the preferred embodiment of the trigger mechanism;

FIGS. 10A and 10B are side elevation views illustrating the syringe observation of an exemplary embodiment of the invention before and after injection;

FIG. 11 are perspective views illustrating the injector final assembly steps;

FIG. 12 is a side elevation view, partially in cross-section, illustrating the syringe assembly;

FIG. 13 are side perspective views, one of which in cross-section, illustrating the exemplary embodiment of the proximal housing;

FIG. 14 is a side perspective view, partially in cross-section, illustrating the exemplary embodiment of the distal housing;

FIG. 15 is a perspective view, in partial cross-section, illustrating the exemplary embodiment of the driver;

FIG. 16 is a perspective view, in partial cross-section, illustrating the exemplary embodiment of the shield with the needle cap remover attached.

### DETAILED DESCRIPTION OF THE INVENTION

The present invention is directed at automatic injectors and needle-locking devices. The injector is automatic in that the needle the needle is automatically inserted into the injection site (e.g., a patient's skin); delivery is automatically initiated upon the insertion of the needle, and the needle is automatically shielded after the end of delivery. The exemplary injectors include features for the delivery of the full content of the syringe independent of component tolerances. Furthermore the shield does not exert a force on the injection site during operation.

The term distal refers to the end or direction of the injector that is applied to an injection site for delivery. The term proximate refers to the end of the injector that is opposite the distal end. The exemplary embodiment shows an injector having a distal end from which the needle is exposed for delivery, and a proximate end opposite the distal end.

Preferably the needle is not seen by the user prior to, during, or after injection. Prior to and after injection, the needle is covered and/or protected by the housing and shield so as to avoid any potential injury or health risk to the user or health care provider.

Without being limited to any particular embodiment, the needle-shielding mechanism can be used in any number of pen-like injectors or other types of injectors or syringes. The needle-shielding mechanism includes a shield latched to the distal housing whereby the shield is unlatched from the distal housing at the end of injection.

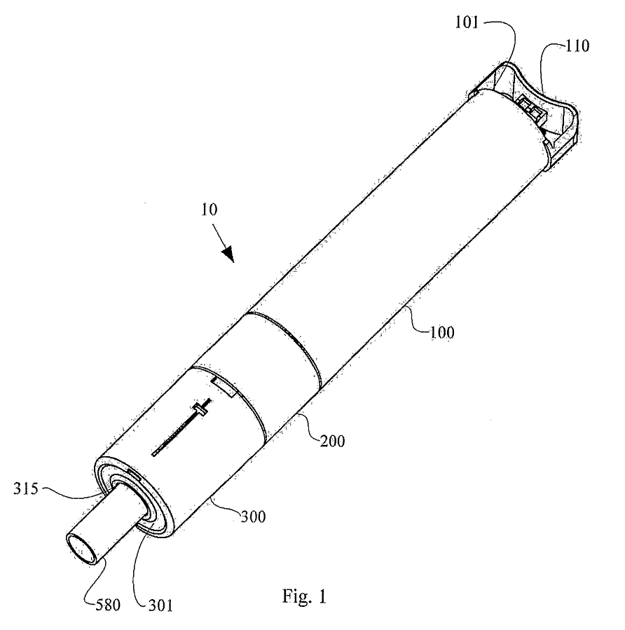

Referring to FIGS. 1-16, there is shown at 10 an automatic injector constructed in accordance with an exemplary embodiment of this invention. In particular, the injector 10 includes a proximal housing 100, a distal housing 200, a shield 300, a driver 400, a cartridge 500, and a shield spring 380 and a driver spring 480. Furthermore the assembly could also include a needle cap remover 580. Externally the automatic injector represents a pen like cylindrical structure as is illustrated in FIG. 1.

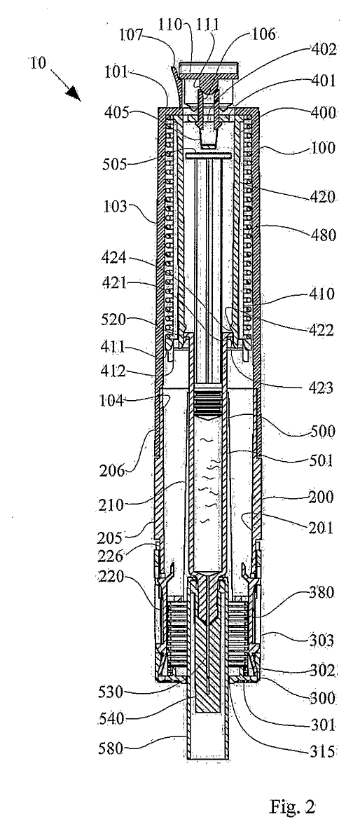

The proximal housing 100 is joined with the distal housing 200 forming an enclosure for the syringe 500, the driver 400 and the driver spring 480 as shown in FIG. 2. The injector 10 has injector trigger 110 positioned on the proximal end 101.

The assembly in a storage position is further illustrated in FIG. 2. The proximal housing 100 and the distal housing 200 have generally cylindrical bodies respectively 103 and 201. These housing halves are joined in the joint section of the proximal housing 104 and joint section of the distal housing 200. The connection of the housing halves could be based on the use of interference fit, adhesives, ultrasound welding or other well known techniques.

The housing is enclosing the driver 400 and the driver spring 480. The base of the driver 401 has a trigger window 402 to accommodate the trigger hooks 106 of the proximal housing. These hooks retain driver 400 in its initial storage position through the coupling to the proximal housing 100. The driver has an integral spring 405. The driver further has a generally cylindrical body 410 with flexible elements 420. These elements along with first protrusions 421 and second protrusions 422 interface with the syringe. The protrusions have a slope on a distal side 423 and 424 to accommodate the assembly and the operation of the device. The distal end of the driver has a rim 411 supporting the compressed driver spring 480 positioned between the driver and the proximal housing.

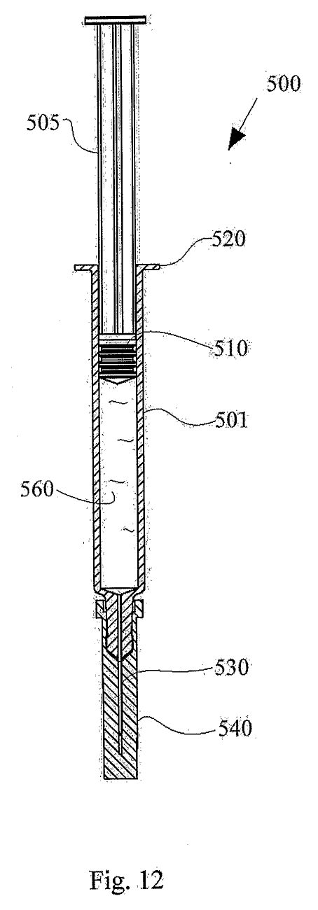

Syringe 500 (see FIG. 12) is also positioned inside the housing. It is supported by the driver flexible elements 420 axially by capturing the flange 520 of the barrel 501 on its narrow side. The syringe is supported radially by the protrusions 210 of the distal housing 200. Preferably, the syringe 500 is a standard, commercially-available, off-the-shelf product available from a plurality of vendors, which may be incorporated into the automatic injector 10 without modification.

The distal housing 200 has a central wall 201 forming a cylindrical body. The base wall 201 has an opening 203 accommodating the syringe barrel 501 and needle cap remover 580. Furthermore the cylindrical body 202 accommodates the shield 300 in a sliding relationship. The distal housing has latches 220 retaining engagement of the shield 300 and the distal housing 200 during storage.

The shield 300 is positioned on the distal housing 200 with the shield spring 380 placed between the distal housing and the shield. The base of the shield 301 has an opening 315 to accommodate the needle cap remover 580. The needle is exposed for delivery at the distal end of the device through the shield opening 315. The shield base 301 has also a rim 302 to center the spring 380. The shield body 303 is arranged to slide on the shield section 226 of the distal housing 200.

The driver 400 and housing 100 have a set of features intended to facilitate the engagement during storage and disengagement of the driver from the housing during triggering. The secure engagement is preventing an accidental release of the driving spring during storage or transportation. The operation of the automatic injector will become clear from detailed description of the automatic injector subassemblies, components and component interactions.

The first step in the use of the automatic injector is the removal of the needle cap 540 of the needle 530 illustrated in the embodiment. The needle cap 540 could consist of one component, an elastomeric protective cap. Alternatively the cap could also include a second component, a rigid plastic cap. The needle cap 540 of the needle 530 protrudes through the distal end of the auto injector (see FIG. 2). It is removed from the injector prior to use. The removal could be assisted by a needle cap remover 580.

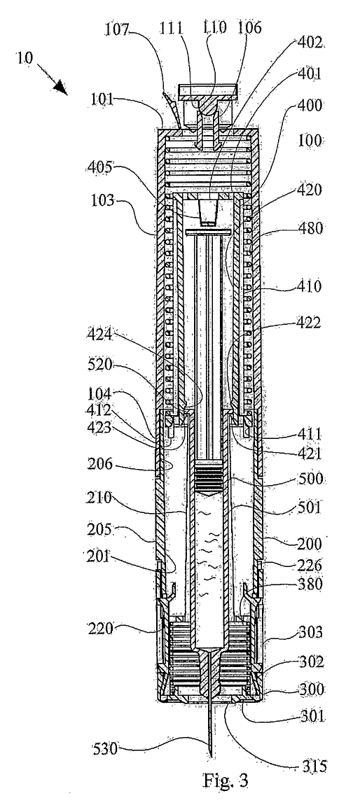

The distal end of the injector is applied to the injection side. The trigger mechanism is actuated by applying an axial force to the trigger 110. The trigger 110 releases the driver 400. The driver 400 is moved in the distal direction by the compressed spring 480. The syringe is retained by the protrusions 421 and 422 and is forwarded by the driver inserting the needle into the subcutaneous tissue. When the needle 530 is fully inserted the syringe flange 520 reaches the distal housing protrusions 210 stopping the forward motion of the syringe. The completion of the needle insertion is illustrated in FIG. 3

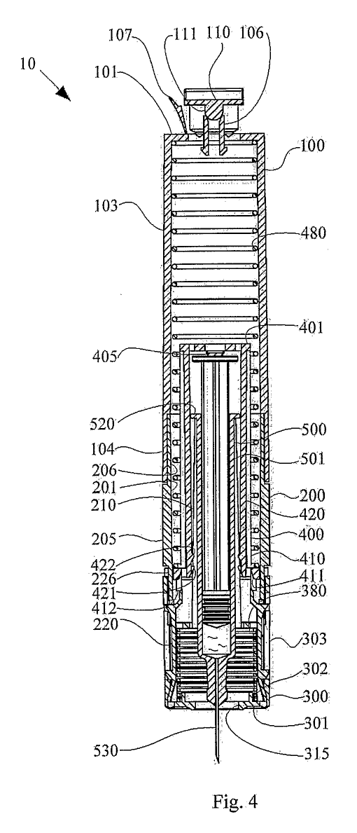

After the syringe flange 520 reaches the distal housing protrusions 210 the radial force component acting on the driver flexible elements 420 increases deflecting the flexible elements 420 radially and allowing the further forward motion of the driver 400. The integral spring 405 of the driver contacts the syringe rod 505 and is deflected as illustrated in FIG. 4. The driver moves rod 505 and stopper 510 and injects the contents of the syringe. The elements 405 along with 420 are acting as a damper to minimize the driver impact on the rod 505 due to the closure of the clearance.

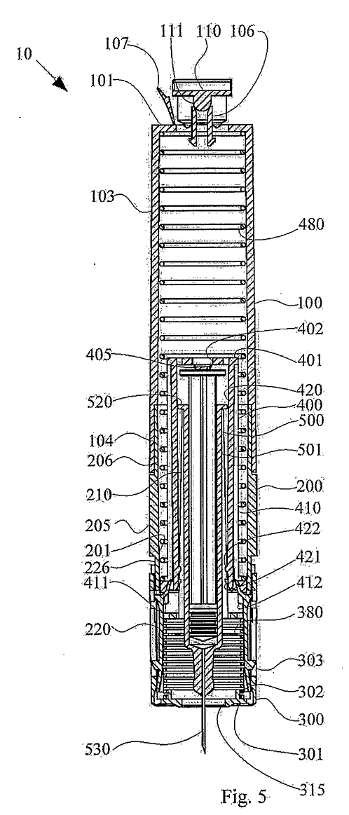

FIG. 5 is a cross-sectional view of the injector at a point when the driver rim 411 reaches the storage latches 220 of the distal housing. The storage latches are disabled by the driver rim 411. At this instance the syringe is either empty or contains a minimal drug volume defined by the tolerances of the system components. In this embodiment the integral compressed spring 405 continues to inject the drug even after the movement of the driver is terminated by the latches.

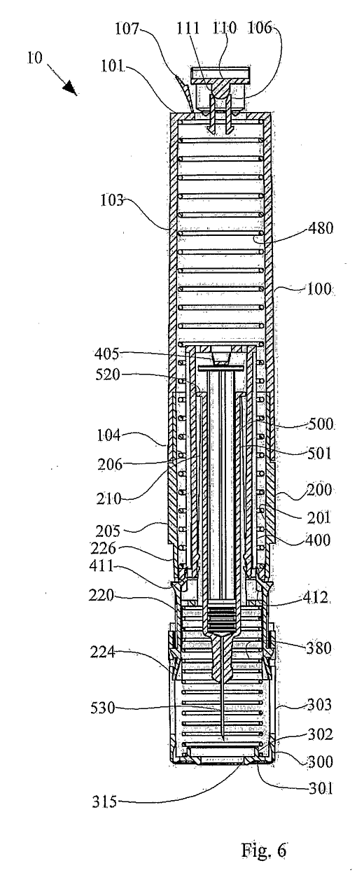

The released shield starts to move under the force of the compressed shield spring 380. The shield applies the force onto the injection site and a reaction force onto the user's hand. Eventually the user allows the shield spring to extract and shield the needle. The time from the shield release to the needle extraction is sufficient to inject the residual drug. An injector with an empty syringe and a shielded needle is illustrated in FIG. 6.

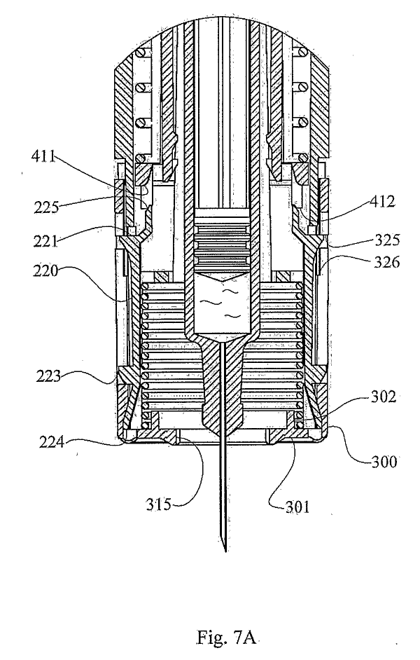

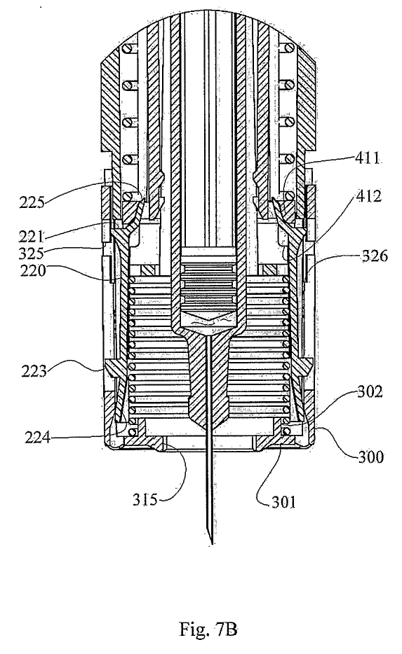

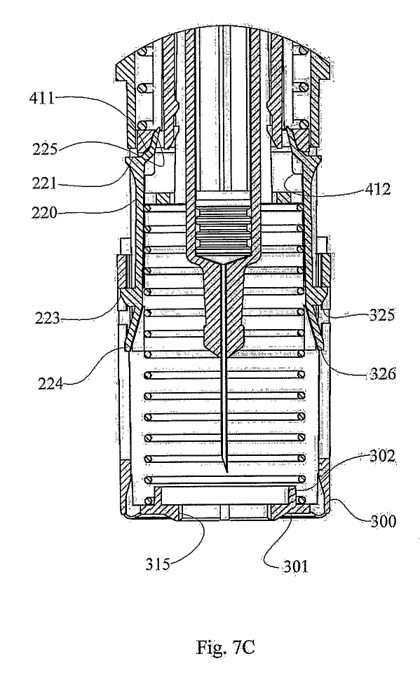

FIGS. 7A-7C illustrate the operation of the shield storage and discard latches in more details. FIG. 7A shows the injector prior to the shield release, FIG. 7B illustrates the shield release, while FIG. 7C illustrates the device with a shielded needle. The storage latches 220 are integral with the distal housing and form a leaf with the base at the storage retention catch 223. The storage retention protrusion 221 of the latch 220 engages the shield bridge 325 during storage. The latch 220 is actuated by the driver rim pushing on the driver rim catch 225. The distal housing also has tabs 224. After the shield is deployed these tabs engage the shield recess edge 326 preventing the shield proximal motion after use in discard position. The tabs 224 are positioned inside the assembly and are not reachable without specialized tooling.

An alternative embodiment of the injector employs body rim fingers 412 to release the shield. In this embodiment the driver has the ability to complete the injection after the shield release is triggered by the fingers 412. The shape of the driver rim catch 225 is modified to accommodate triggering of the shield release by the rim fingers 412 prior to the driver rim contacting the driver rim catch 225. The driver continues to move to inject the residual drug, if any, after the shield is released.

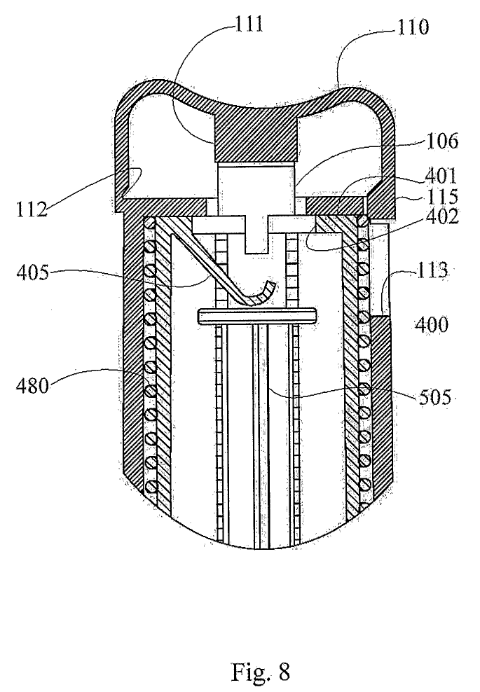

The integral spring of the driver is illustrated in FIG. 8. The figure shows the spring cross section as molded. It is deflected only during injection and is injecting the residual drug after the driver stops moving. This figure also illustrates some of the features of the trigger mechanism. The trigger is molded integral with the proximal housing and has a living hinge 112. The opposite trigger end 115 is accommodated by a recess in the proximal housing 113.

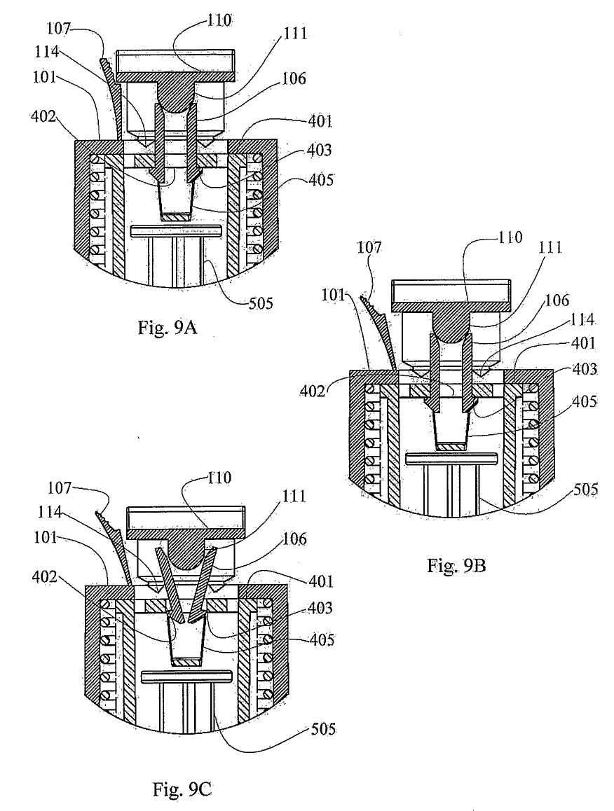

The trigger mechanism is further illustrated in FIGS. 9A-9C. The trigger has a safety latch 107 preventing triggering as illustrated in FIG. 9A. The user has to bend this latch prior to operation as shown in FIG. 9B to enable triggering. The protrusions 111 spread the retaining hooks 106 when the trigger 110 is depressed. As a result the driver is released as shown in FIG. 9C. The location of the hook pivot 114 outside of the driver trigger window edge 403 engaging the trigger hooks 106 leads to stable operation in storage.

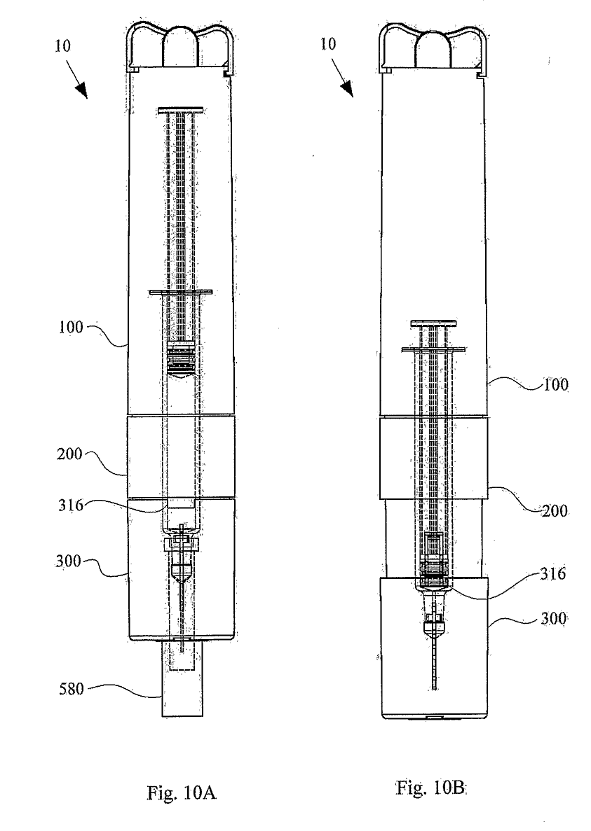

The distal housing is made from a clear plastic and/or with windows to provide the user the ability to see dosage formulation prior to use, and visually confirm the drug has been delivered. FIGS. 10A and 10B illustrate the syringe observation before and after injection of an exemplary embodiment of the invention. The distal housing could be made from a clear plastic resulting in a visible syringe in storage illustrated in FIG. 10A. A cutout 316 could be provided on the shield 300 for a better observation of the syringe. Furthermore the cutout assist the visualization of the end of the syringe barrel with a stopper 510 confirming the complete drug delivery as illustrated in FIG. 10B.

In the exemplary embodiment, the injector deploys its needle automatically, delivers the drug in the syringe and shields the needle automatically. Preferably the injector provides distinct audible indications (e.g., a ‘click-type’ effect) and a tactile feedbacks on the injection progress.

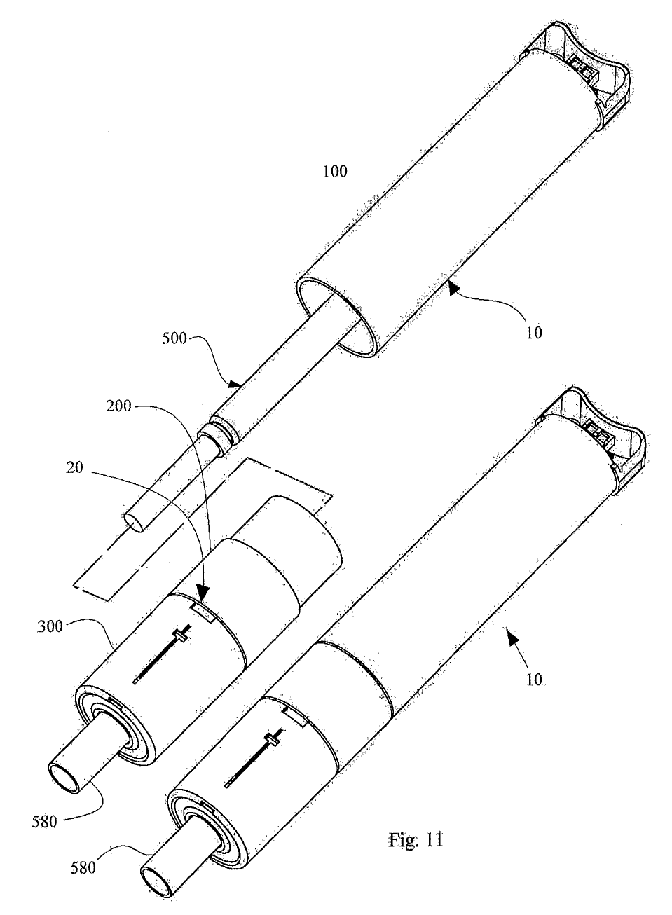

The injector can be delivered to the final assembly with a syringe in two subassemblies as illustrated in FIG. 11. One subassembly 15 integrates the proximal housing with the driver and the driver spring. The second subassembly 20 integrates the distal housing with the shield, needle cap remover 580 and the shield spring 380. These two subassemblies are integrated with the syringe during the final assembly stage.

A typical syringe 500 is illustrated in FIG. 12. It has a glass barrel 501 with a staked needle 530. The glass barrel 501 has a flange 520. The barrel is filled with a drug 560. The drug is sealed by a stopper 510 which is in a sliding relationship with the barrel. The needle is shielded by a needle cap 540 made from an elastomeric material. The needle cap 540 frequently is an assembly with a rigid plastic cap. The syringe is provided with a rod 505 to push the stopper 510 to inject the drug 560.

Alternatively the cartridge could have a double sided needle (as in Carpuject cartridge). The needle would require an axial force at the beginning of operation to activate the cartridge and to cause the penetration of the proximate end of the needle through the rubber stopper for drug delivery.

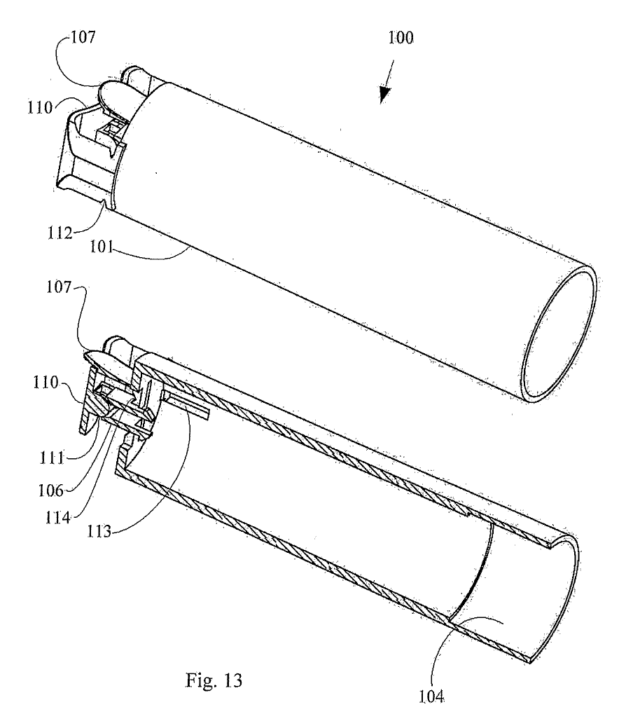

FIG. 13 illustrates the exemplary embodiment of the proximal housing. The trigger could be molded integral with the housing. FIG. 13 illustrates the proximal housing after the trigger has been rotated and snapped to engage the trigger recess 113.

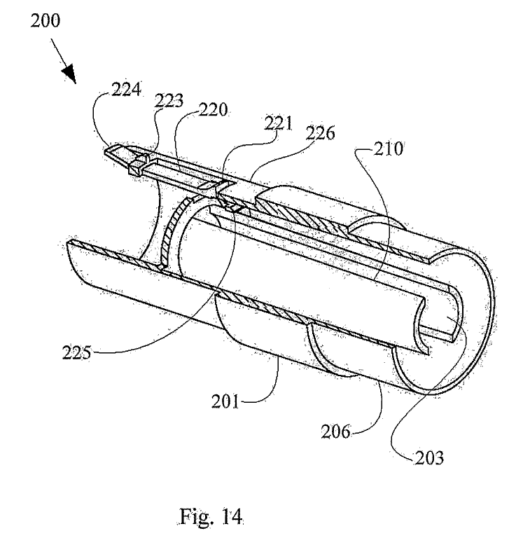

FIG. 14 illustrates the exemplary embodiment of the distal housing. This part could be molded from a clear plastic such as polycarbonate and could additionally have observation windows.

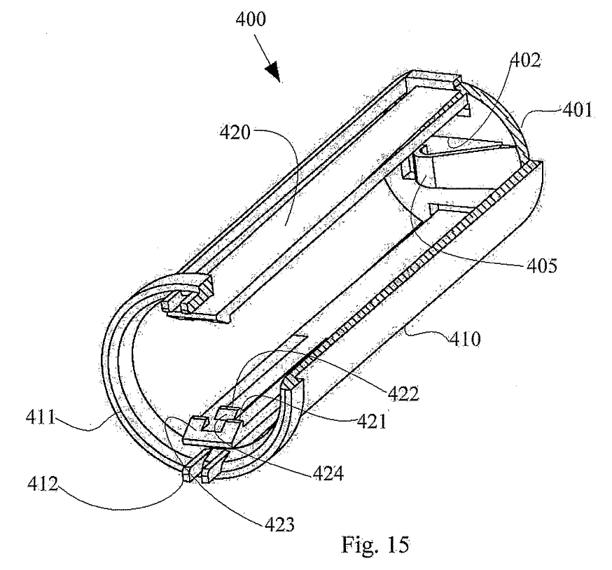

FIG. 15 illustrates the exemplary embodiment of the driver. The leading slopes of the second protrusions 424 and the spring properties of the flexible elements 420 are selected to achieve a required force for driver disengagement from the syringe during operation.

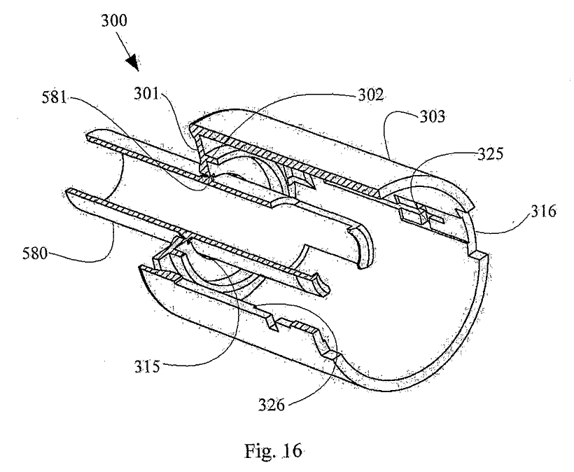

FIG. 16 illustrates the exemplary embodiment of the shield with the needle cap remover attached by bridges 581. These bridges retain the needle cap remover 580 integral with the shield 300 during the subassembly process. The needle cap remover bridges could be fractured or removed during the final assembly stage.

It should be apparent from the aforementioned description and attached drawings that the concept of the present application may be readily applied to a variety of preferred embodiments, including the exemplary embodiments disclosed herein. For example, other driving and retraction units, such as elastomeric “O” rings or compressed gas may be used in place of the compression springs disclosed herein to bias the driver. It will be appreciated by those skilled in the art that changes could be made to the embodiment described above without departing from the broad inventive concept thereof. It is understood, therefore, that this invention is not limited to the particular embodiments disclosed, but it is intended to cover foreseeable modifications within the spirit and scope of the present invention as defined by the appended claims.

It is further appreciated that the present invention may be used to deliver a number of drugs. The term “drug” used herein includes but is not limited to peptides or proteins (and mimetic thereof), antigens, vaccines, including DNA vaccines, hormones, analgesics, anti-migraine agents, anti-coagulant agents, medications directed to the treatment of diseases and conditions of the central nervous system, narcotic antagonists, immunosuppressants, agents used in the treatment of AIDS, chelating agents, anti-anginal agents, chemotherapy agents, sedatives, anti-neoplastics, prostaglandins, antidiuretic agents and DNA or DNA/RNA molecules to support gene therapy.

Typical drugs include peptides, proteins or hormones (or any memetic or analogues of any thereof) such as insulin, calcitonin, calcitonin gene regulating protein, atrial natriuretic protein, colony stimulating factor, betaseron, erythropoietin (EPO), interferons such as alpha., .beta., or gamma. interferon, somatropin, somatotropin, somastostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues or antagonists thereof, such as IL-1ra, thereof, analgesics such as fentanyl, sufentanil, butorphanol, bup renorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; antiemetic agents such as scopolamine, ondansetron, domperidone, metoclopramide, and analogues thereof; cardiovascular agents, anti-hypertensive agents and vasodilators such as diltiazem, clonidine, nifedipine, verapamnil, isosorbide-5-mononitrate, organic nitrates, agents used in treatment of heart disorders, and analogues thereof; sedatives such as benzodiazepines, phenothiozines, and analogues thereof; chelating agents such as deferoxamine, and analogues thereof; anti-diuretic agents such as desmopressin, vasopressin, and analogues thereof; anti-anginal agents such as nitroglycerine, and analogues thereof; anti-neoplastics such as fluorouracil, bleomycin, and analogues thereof; prostaglandins and analogues thereof; and chemotherapy agents such as vincristine, and analogues thereof, treatments for attention deficit disorder, methylphenidate, fluoxamine, Bisolperol, tactolimuls, sacrolimus and cyclosporin.

Without further elaboration, the foregoing will so fully illustrate the invention that others may, by applying current or future knowledge, readily adapt the same for use under various conditions of service.

What is claimed is:

1. An automatic injector for delivering a dose of medicament into a user's body at an injection site, comprising:
a housing having a proximal end and a distal end;
a syringe assembly movably supported within the housing, the syringe assembly including a syringe barrel having an open end and a closed end, a plunger assembly including a stopper, and an injection needle;
a drive member operably and releasably coupled with the syringe barrel and movable between a retracted position and an extended position;
a drive spring operably coupled to the drive member to bias the drive member into the extended position;
a trigger mechanism including a latch capable of retaining the drive member in the retracted position against a force of the drive spring;
at least one mechanical stop connected to the housing, limiting movement of the syringe assembly toward the distal end of the housing to a maximally extended position;
wherein:
activation of the trigger mechanism causes movement of the latch to release the drive member retained in the retracted position, and the drive spring forces the drive member, and the syringe assembly releasably coupled thereto, to move toward the distal end of the housing from the retracted position toward the extended position, forcing the needle to puncture the user's body at the injection site;
as the needle punctures the user's body, a resistive force acts upon the needle, and upon one of the syringe barrel contacting the mechanical stop and resistive force reaching a predetermined level, the drive member disengages the syringe barrel and additional movement of the syringe barrel and the needle coupled thereto in the direction of the distal end of the housing is prevented; and
with the drive member disengaged from the syringe barrel, the drive spring forces continued movement of the drive member in the direction of the distal end of the housing, forcing the drive member into engagement with the syringe plunger assembly, and forcing the plunger assembly to expel the medicament contained within the syringe barrel through the needle into the user's body.

2. The automatic injector of claim 1, wherein the drive member extended position corresponds to engagement of the stopper with the closed end of the syringe barrel, and wherein the drive member drivingly engages the plunger assembly until the drive member reaches the extended position.

3. The automatic injector of claim 1, the syringe barrel including a flange and the drive member including a protrusion engaging the flange, wherein upon application of the resistive force of a predetermined level, the flange rides over the protrusion to disengage the syringe barrel from the drive member.

4. The automatic injector of claim 3, wherein with the syringe assembly in the maximally extended position, the at least one mechanical stop engages the syringe flange to prevent further movement of the syringe assembly toward the distal end of the housing.

5. The automatic injector of claim 1, further comprising a dampening spring operably coupled to one of the drive member and the syringe plunger and positioned between the drive member and the syringe plunger.

6. The automatic injector of claim 5, wherein as the drive member moves into the extended position, the stopper is separated from the closed end of the syringe barrel, and the dampening spring drivingly engages the plunger assembly to force the stopper into engagement with the closed end.

7. The automatic injector of claim 1, wherein the housing is formed from a cylindrical proximal housing portion and a cylindrical distal housing portion fixedly joined together.

8. The automatic injector of claim 1, wherein the housing is fabricated from a substantially clear material allowing the user to see through the housing.

9. The automatic injector of claim 1, further comprising at least one window formed in the housing providing the user with visual access to an interior of the housing.

10. The automatic injector of claim 1, the trigger mechanism further comprising a safety latch capable of being manually bent from a latching position wherein the latch prevents activation of the trigger mechanism into an activated position wherein the latch does not prevent activation of the trigger mechanism.

11. The automatic injector of claim 1, further comprising a needle cap remover extending from the distal end of the housing to facilitate manual removal of a needle cap.

12. The automatic injector of claim 1, wherein the drive spring and the syringe barrel are arranged coaxially within the housing.

13. The automatic injector of claim 1, wherein the syringe assembly is a standard, commercially-available, off-the-shelf syringe assembly.

14. An automatic injector for delivering a dose of medicament into a user's body at an injection site, comprising:
a housing having a proximal end and a distal end;
a syringe assembly movably supported within the housing, the syringe assembly including a syringe barrel adapted to contain the medicament, a plunger assembly, and an injection needle;
a drive member operably and releasably coupled with the syringe barrel and movable between a drive member retracted position and a drive member extended position;
a drive member drive spring operably coupled to the drive member to bias the drive member in the drive member extended position;
a trigger mechanism including a drive member latch capable of retaining the drive member in the drive member retracted position against the force of the drive member drive spring;
a shield operably and movably coupled to the distal end of the housing and movable between a shield retracted position and a shield extended position;
a shield drive spring operably coupled to the shield to bias the shield member into the shield extended position;
a first shield latch adapted to engage the shield and capable of retaining the shield in the shield retracted position against the force of the shield drive spring;
wherein
activation of the trigger mechanism causes movement of the drive member latch to release the drive member retained in the drive member retracted position, and the drive member drive spring forces the drive member, and the syringe assembly releasably coupled thereto, to move toward the distal end of the enclosure from the drive member retracted position toward the drive member extended position, forcing the needle to puncture the user's body at the injection site;
at a position intermediate the drive member retracted position and the drive member extended position, the drive member contacts the first shield latch to move the first shield latch to release the shield from the shield retracted position; and
the shield spring moves the shield from the shield retracted position to the shield extended position as the drive member drive spring moves the drive member from the drive member intermediate position to the drive member extended position.

15. The automatic injector of claim 14, further comprising a second shield latch adapted to engage the shield positioned in the shield extended position and to retain the shield in the shield extended position.

16. The automatic injector of claim 14, wherein as the shield spring moves the shield from the shield retracted position to the shield extended position, the shield is moved toward the housing distal end, urging the shield toward the user's body with a force of the shield spring being sufficient to extract the needle from the user's body.

17. The automatic injector of claim 16, wherein the drive member, drive member drive spring, syringe assembly, shield and shield drive spring are configured such that substantially all of the medicament contained within the syringe barrel is injected into the user's body before the needle is extracted from the user's body.

18. The automatic injector of claim 17, wherein the drive member intermediate position is sufficiently proximate the extended position that there is substantially no force applied to the user's body by the shield spring during injection of the medicament into the user's body.

19. The automatic injector of claim 14, wherein the shield spring and the shield are arranged coaxially within the housing.

20. The automatic injector of claim 14, wherein the syringe assembly is a standard, commercially-available, off-the-shelf syringe assembly.

21. An automatic injector for delivering a dose of medicament into a user at an injection site, comprising:
a housing having a proximal end and a distal end;
a syringe assembly movably supported within the housing, the syringe assembly including a syringe barrel adapted to contain the medicament, a plunger assembly, and an injection needle;
a drive member operably and releasably coupled with the syringe barrel and movable between a drive member retracted position and a drive member extended position;
a drive member drive spring operably coupled to the drive member to bias the drive member in the drive member extended position;
a trigger mechanism including a drive member latch capable of retaining the drive member in the drive member retracted position against the force of the drive member drive spring;
a shield operably and movably coupled to the distal end of the housing and movable between a shield retracted position and a shield extended position;
a shield drive spring operably coupled to the shield to bias the shield member into the shield extended position;
a first shield latch adapted to engage the shield and capable of retaining the shield in the shield retracted position against the force of the shield drive spring;
at least one mechanical stop connected to the housing, limiting movement of the syringe assembly toward the distal end of the housing to a maximally extended position;
wherein:
activation of the trigger mechanism causes movement of the drive member latch to release the drive member retained in the drive member retracted position, and the drive member drive spring forces the drive member, and the syringe assembly releasably coupled thereto, to move toward the distal end of the enclosure from the drive member retracted position toward the drive member extended position, forcing the needle to puncture the user's body at the injection site;
as the needle punctures the user's body, a resistive force acts upon the needle, and upon one of the syringe barrel contacting the mechanical stop and resistive force reaching a predetermined level, the drive member disengages the syringe barrel and additional movement of the syringe barrel and the needle coupled thereto in the direction of the distal end of the housing is prevented;
with the drive member disengaged from the syringe barrel, the drive member drive spring forces continued movement of the drive member in the direction of the distal end of the housing, forcing the drive member into engagement with the syringe plunger assembly, and forcing the plunger assembly to expel the medicament contained within the syringe barrel through the needle into the user's body until the drive member reaches the extended position;
at a position intermediate the drive member retracted position and the drive member extended position, the drive member contacts the first shield latch to move the first shield latch to release the shield from the shield retracted position; and
the shield spring moves the shield from the shield retracted position to the shield extended position as the drive member drive spring moves the drive member from the drive member intermediate position to the drive member extended position.

22. A method of manufacturing an automatic injector for delivering a fluid, comprising the steps of:
providing a cylindrical proximal housing assembly including a driver member, a driver spring, and a trigger mechanism having a latch, the driver member being retained in a retracted position by the latch;
providing a cylindrical distal housing assembly;
installing a syringe assembly within one of the proximal housing assembly and the distal housing assembly; and
connecting the distal housing assembly to the proximal housing assembly to form an enclosure at least substantially enclosing the syringe assembly such that the syringe assembly is operably engaged with the driver member.

23. The method of manufacturing an automatic injector of claim 22, wherein the step of connecting the distal housing to the proximal housing is accomplished using one of an interference fit, adhesive, and ultrasonic welding.

24. The method of manufacturing an automatic injector of claim 23, wherein the syringe assembly is a standard, commercially-available, off-the-shelf syringe assembly.
